(19) United States
(12) Patent Application Publication
(10) Pub. No.: US 2005/0232435 A1
(43) Pub. Date: Oct. 20, 2005

(54) Noise attenuation system for vehicles

(76) Inventors: Ian McGregor Stothers, Saham Tomey (GB); Ivan Anthony Scott, Cambridge (GB); Stephen John Elliott, Winchester (GB)

Correspondence Address: SMITH, GAMBRELL & RUSSELL, LLP, 1230 PEACHTREE STREET, N.E., SUITE 3100, PROMENADE II, ATLANTA, GA, 30309-3592, US

(21) Appl. No.: 11/156,158

(22) Filed: Jun. 17, 2005

Related U.S. Application Data
(63) Continuation of application No. PCT/GB03/05481, filed on Dec. 16, 2003.

(30) Foreign Application Priority Data
Dec. 19, 2002 (GB) GA 0229580.6

(51) Int. Cl. A61F 11/06; G10K 11/16; H03B 29/00

(52) U.S. Cl. 381/71.11; 381/71.4; 381/71.7

(57) ABSTRACT

A system for attenuating noise in a cabin of a vehicle is provided in which the vehicle has an interior wall defining the cabin and an exterior wall defining a cavity between the interior wall and the exterior wall. A plurality of actuators are arranged within the cavity, on the interior wall or on the exterior wall for providing a physical actuation resulting in an acoustic disturbance. A plurality of sensors are arranged in the cavity on the interior wall, or on the exterior wall to provide indications of acoustic disturbances. A control system uses the indications from the sensors to generate control signals for the actuators. In one arrangement the sensors are arranged in an array interspersed between the actuators which are also arranged in an array. A drive signal for each actuator is then generated using the signals from sensors adjacent to the actuators.

### CROSS REFERENCE TO RELATED APPLICATIONS

This application is a continuation of International Application PCT/GB2003/005481 with an International Filing Date of Dec. 16, 2003, and claiming priority to co-pending Great Britain Patent Application No. 0229580.6 filed on Dec. 19, 2002, both of which are relied on and incorporated herein by reference.

The present invention generally relates to noise attenuation systems for reducing noise within a cabin of a vehicle.

In passenger carrying vehicles there is a well known problem of noise generated by engine or engines of the vehicle as well as other noise factors such as wind noise. The noise generated externally to the vehicle cabin can make the journey for the passengers within the cabin unpleasant. This problem is particularly acute in aircraft where engine noise and the noise of the air passing over the fuselage is a particular problem. There are two methods of addressing the problem which can be used in combination. One method is to use passive means such as sound absorbing material or stiffer structures. Another method is to use active noise control in which vibrations are cancelled by injecting vibrations of equal and opposite phase into the structure or cabin. The problem of active noise control has been widely addressed in the prior art in many different ways.

Active noise control using adaptive signal processing is widely known in the prior art (see for example “Adaptive Signal Processing” by B Widrow and S. D. Stearns, Prentice Hall Signal Processing Series, 1985), the content of which is hereby incorporated by reference. One technique that has been widely applied to active noise control is the least mean square (LMS) algorithm for the adaption of the filter coefficients in the adaptive filter used in adaptive control. WO88/02912 and WO95/26521 disclose the use of the LMS algorithm for multiple sensors and multiple actuators. The contents of WO88/02912 and WO95/26521 are herby incorporated by reference. WO88/02912 discloses a method of reducing noise within a motor car passenger compartment. This is achieved by placing a plurality of microphones and loudspeakers within the passenger compartment. The system requires a complex control system in view of the need to calculate filter coefficients for all of the cross terms in the matrix i.e. to calculate coefficients for all microphones and all loudspeakers because the system tries to cancel the noise within the volume of the passenger compartment.

A digital virtual earth cancellation system for cancelling noise is disclosed in WO91/12579, the content of which is hereby incorporated by reference. In this arrangement because the generation of the cancellation signal causes the generation of a cancelling sound near to the residual signal detection, there is a feedback signal which needs to be compensated for. This is achieved using a feedback removal path comprising a filter providing a model of the path between the cancellation signal generation and the residual signal detection.

In one prior art arrangement disclosed in U.S. Pat. No. 6,078,673, a simple system of independent noise cancelling cells in the cavity between the external fuselage and the internal cabin wall is disclosed. In this arrangement independent virtual earth circuits provide for independent cancellation of noise in a cell region. Each virtual earth circuit comprises a loudspeaker, an acoustic sensor and a feedback circuit. The sound detected by the acoustic sensor is fed back through the feedback circuit in an inverted form to drive the loudspeaker. Thus this arrangement provides a simple system in which each cell acts independently.

It is an object of the present invention to provide an improved noise cancelling system. In particular it is an object of one aspect of the present invention to provide a simple yet improved system for cancelling noise in a vehicle cabin.

In one aspect the present invention provides a system for attenuating noise in a cabin of a vehicle by providing the noise attenuation components within or associated with a cavity between an interior wall defining the cabin and an exterior wall of the vehicle.

In one aspect of the present invention, the system comprises a plurality of actuators and a plurality of sensors arranged in the cavity such as in a common plane which need not be planar and can be contoured, on or adjacent to the interior wall so as to provide a panel-like arrangement of actuators and sensors for reducing noise and vibrations transmitted into the cabin through the cabin wall. A control system is connected to the actuators and sensors and controls each actuator in dependence on signals received from sensors adjacent to the actuator.

Thus in this aspect of the present invention by arranging the actuator and sensors in a common plane in the cavity or on the interior or exterior walls and by using the detections by the sensors adjacent to each actuator to control each respective actuator, noise and/or vibrations can be effectively reduced. In one embodiment, where the array of sensors and actuators are provided in a plane parallel to the interior wall, the nearest neighbour sensors to each actuator detect vibrations and noise propagating along the cabin wall or cavity and provide an advanced warning i.e. a feedforward signal. The sensors also provide a detection of the residual vibration or noise i.e. a feedback signal.

This aspect of the present invention is particularly useful for cancelling noise propagating along the cabin wall of the vehicle. Examples of such noise are wind noise caused by air flow along the outer wall of the vehicle, and jet outlet noise in aircraft.

In this aspect of the present invention, by using signals from a plurality of sensors adjacent to each actuator for controlling the actuator to generate a noise reducing acoustic disturbance, a more accurate noise cancelling system is produced. The present invention is not limited to the use of only the nearest neighbour sensors next to each actuator for the control of the actuator. In a more complex embodiment of the present invention, next nearest neighbour or even third nearest neighbour sensors could be used to control the actuation of an actuator. By using only a limited number of nearest neighbour sensors in this way important cross coupling factors are taken into account without having to take into account all cross coupling factors between the array of sensors and actuators.

In one embodiment of the present invention, neighbouring sensors are used to control an actuator by taking a sum or weighted sum of the signals from the sensors to generate a drive signal for the actuator. The summation can be filtered in order to provide a suitable drive signal for the actuator. This provides a simple feedback system wherein attenuating “cells” are not independent of each other but overlap by the incorporation of common sensors in adjacent “cells”. Thus in this way the distance between cells need not be large so as to avoid cross-talk.

In another embodiment of the present invention the control system performs adaptive filtering of the signals from the sensors in order to generate the drive signal for the actuator. Any suitable adaptive control system can be used. In a preferred embodiment the adaptive control system uses the least mean square algorithm for adapting the filtering. The least mean square algorithm can use not only the signals from the sensors adjacent to the actuator for generating the drive signal for the actuator, but also the drive signals for neighbouring actuators to adaptively determine the filtering to be performed to generate the drive signal for the actuator.

In one preferred embodiment of the present invention at least one reference sensor is arranged away from the array of actuators and the array of sensors towards the exterior wall to provide at least one reference detection indicative of acoustic disturbances propagating across the cavity towards the array of actuators and sensors. A control system uses the or each reference detection in the generation of the drive signal for the or each actuator so that in this embodiment of the present invention there is a feedforward signal indicative of acoustic disturbances propagating across the cavity as well as feedforward signals provided by sensors within the array indicative of acoustic disturbances propagating along the cavity or interior wall. In one embodiment there is provided an array of reference sensors arranged towards the exterior wall such that the array of reference sensors are arranged substantially parallel to the array of actuators and sensors. Thus, in this manner, each actuator can be provided with feedforward signals for acoustic disturbances propagating across the cavity as well as along the cavity. This greatly enhances the efficiency of the system because advance information on the propagating noise is made available to allow for advanced processing and calculation of cancelling signals.

Another aspect of the present invention provides a system for attenuating noise in a cabin of a vehicle. The vehicle has an interior wall defining the cabin and an exterior wall defining a cavity between the interior wall and the exterior wall. A plurality of actuators are arranged in the cavity, on the interior wall, or on the exterior wall for providing a physical actuation resulting in an acoustic disturbance. A plurality of sensors are also arranged in the cavity, on the interior wall, or on the exterior wall for providing signals indicative of acoustic disturbances. Control means for generating a drive signal for each actuator uses the signals from the sensors adjacent to each respective actuator in order to generate the drive signal.

Another aspect of the present invention provides a system for attenuating noise in the cabin of a vehicle in which the vehicle has an interior wall defining the cabin and an exterior wall defining the cavity between the interior wall and the exterior wall. At least one reference detector is arranged on or towards the exterior wall for providing at least one reference signal indicative of acoustic disturbances. At least one actuator is arranged on or towards the interior wall for providing at least one physical actuation resulting in at least one acoustic disturbance. At least one residual detector is arranged on or towards the interior wall for providing at least one residual signal indicative of residual acoustic disturbances. Control means are provided for using each reference signal and each residual signal in the generation of a drive signal for each actuator.

Thus, this aspect of the present invention provides a system in which feedforward and feedback signals are used for the generation of a drive signal for attenuating noise in the cabin of a vehicle.

Another aspect of the present invention provides a system for attenuating noise in the cabin of an aircraft, wherein the aircraft has an interior wall defining the cabin and an exterior fuselage defining a cavity between the interior wall and the exterior fuselage. At least one actuator is arranged in the cavity, on the interior wall, or on the exterior wall for providing at least one physical actuation resulting in at least one acoustic disturbance. At least one sensor is arranged in the cavity, on the interior wall, or on the exterior wall for providing at least one indication of acoustic disturbances. A control system is provided for using the indications from each sensor to generate a control signal for each actuator. A switch mode power amplifier means is provided for amplifying each generated control signal to generate drive signal for each actuator.

The use of a switch mode power amplifier means overcomes a significant problem in providing an active noise control system in an aircraft. The dissipation of heat in an aircraft is a significant problem. By providing an active noise control system, amplifiers are required in order to provide a significant energy input in order to attenuate noise. This significant energy input requires significant power amplification in order to drive the actuators. The present inventors have realized that by choosing to use a switch mode power amplifier arrangement for amplifying each of the signals to the actuators, there is a significant reduction in heat generation and there is no need for heat sinks. This saving in the use of heat sinks is a significant weight reduction which has obvious advantages in aircraft, the most significant being a fuel saving.

Embodiments of the present invention will now be described with reference to the accompanying drawings, in which:

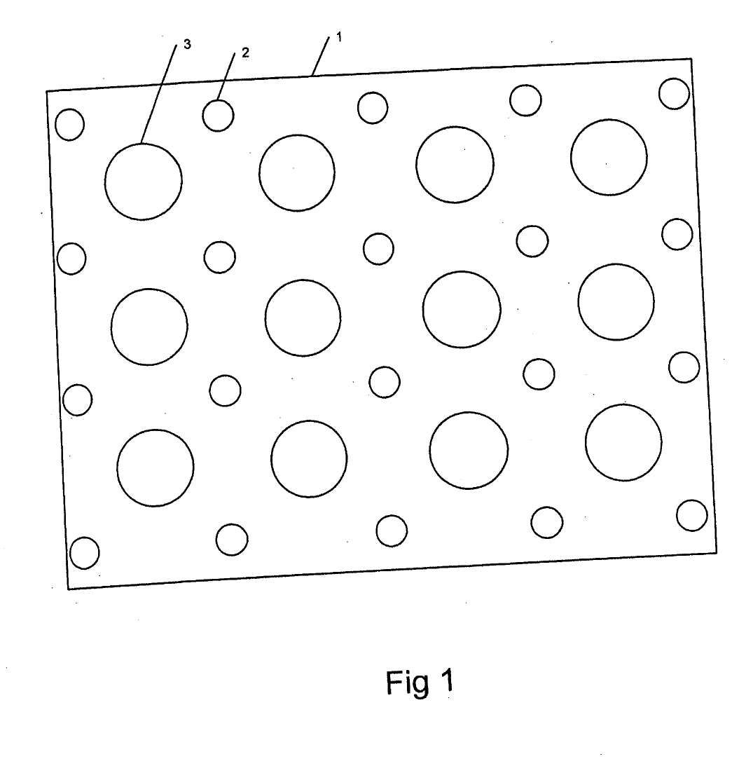

FIG. 1 is a plan view of a noise attenuating panel;

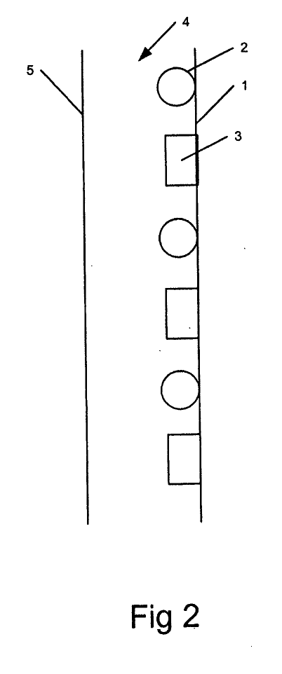

FIG. 2 is a schematic cross-sectional view of the arrangement of the panel of FIG. 1 in the cavity of a vehicle such as an aircraft;

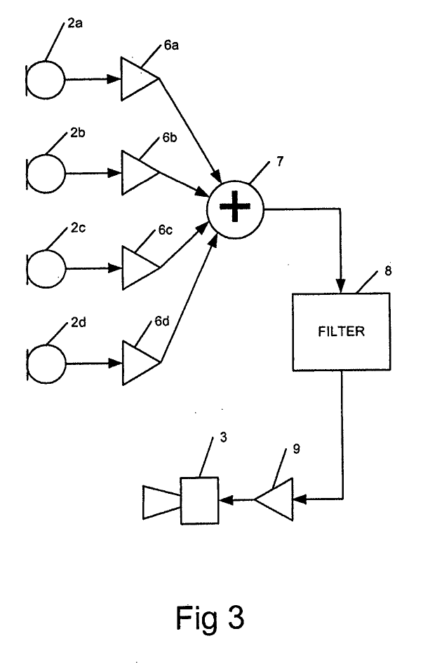

FIG. 3 is a schematic diagram of an active noise control unit for generating a drive signal for an actuator in accordance with an embodiment of the present invention;

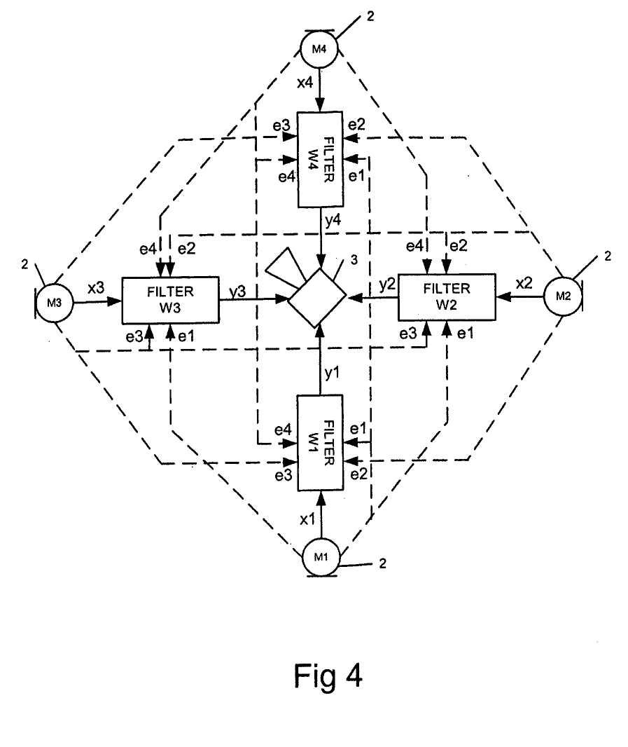

FIG. 4 is a schematic diagram of an active noise control unit for generating a drive signal for an actuator in accordance with another embodiment of the present invention;

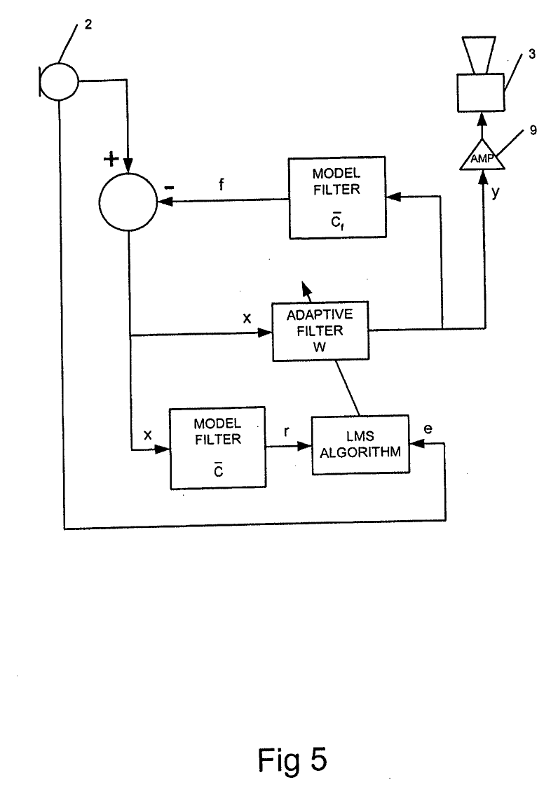

FIG. 5 is a schematic diagram of one channel of the embodiment of FIG. 4;

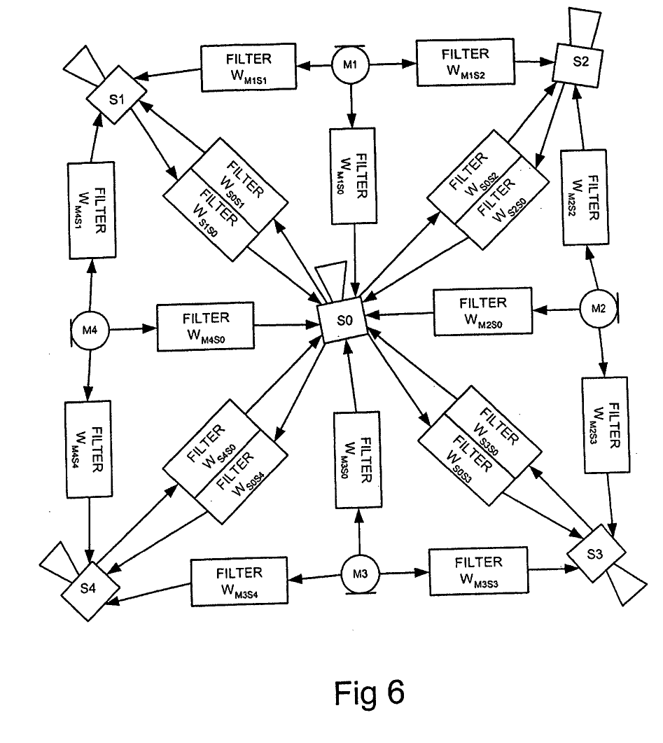

FIG. 6 is a schematic diagram of an active noise control unit for generating a drive signal for an actuator in accordance with a further embodiment of the present invention;

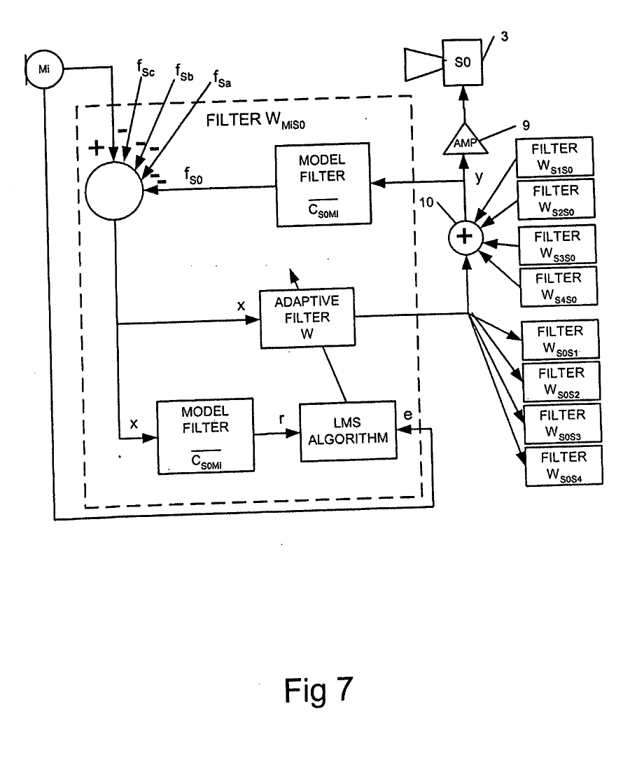

FIG. 7 is a schematic diagram of a single channel for the adaptive filter W_{MiS0} in the embodiment of FIG. 6;

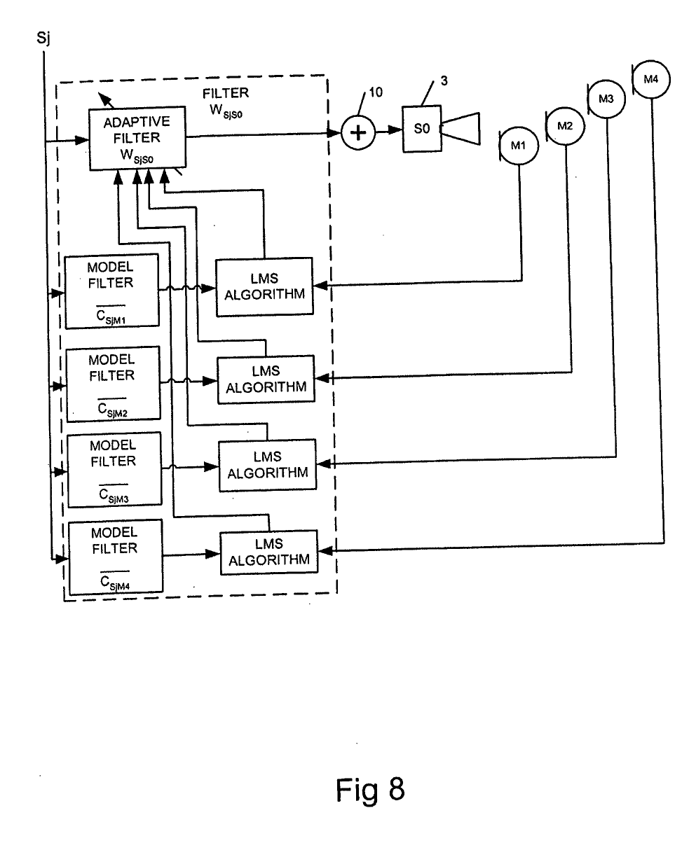

FIG. 8 is a schematic diagram of a single channel for generating a drive signal for the loudspeaker SO based on the drive signal for adjacent loudspeakers Sj using the adaptive filter W_{SjS0} in the embodiment of FIG. 6 assuming that the acoustic model of the paths between the microphones M1, M2, M3 and M4 adjacent to the loudspeaker SO are different;

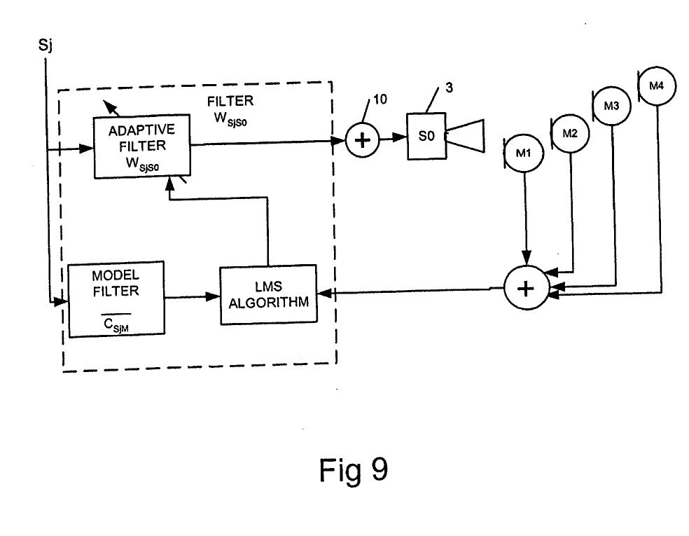

FIG. 9 is a schematic diagram of a single channel for generating a drive signal for the loudspeaker SO based on the drive signal for adjacent loudspeakers Sj using the adaptive filter W_{SjS0} in the embodiment of FIG. 6 assuming that the acoustic model of the paths between the microphones M1, M2, M3 and M4 adjacent to the loudspeaker SO are the same;

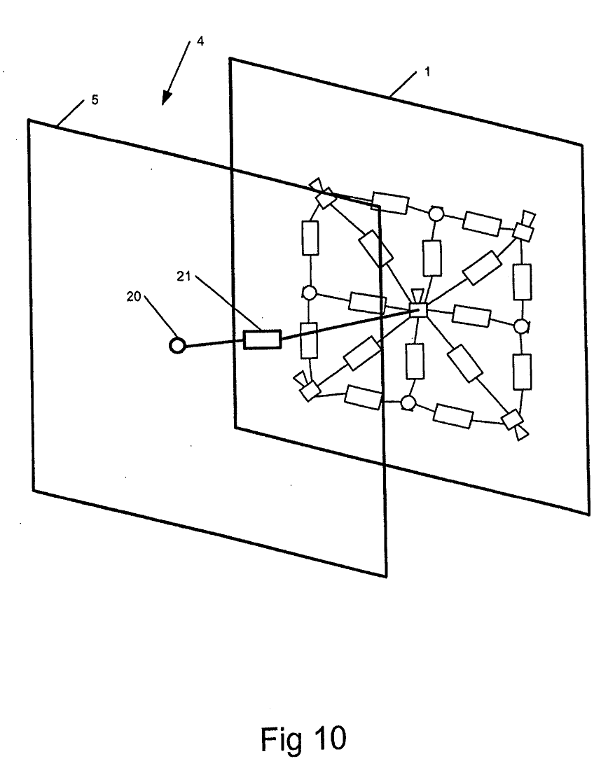

FIG. 10 is a schematic diagram of the application of the active noise control unit to a panel in a cavity of an aircraft and the addition of a feedforward sensor, in accordance with an embodiment of the present invention;

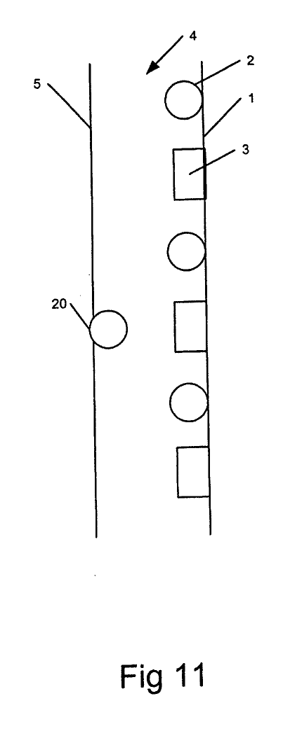

FIG. 11 is a schematic cross-sectional view of the arrangement of the panel in FIG. 10 in the cavity of the vehicle, such as an aircraft;

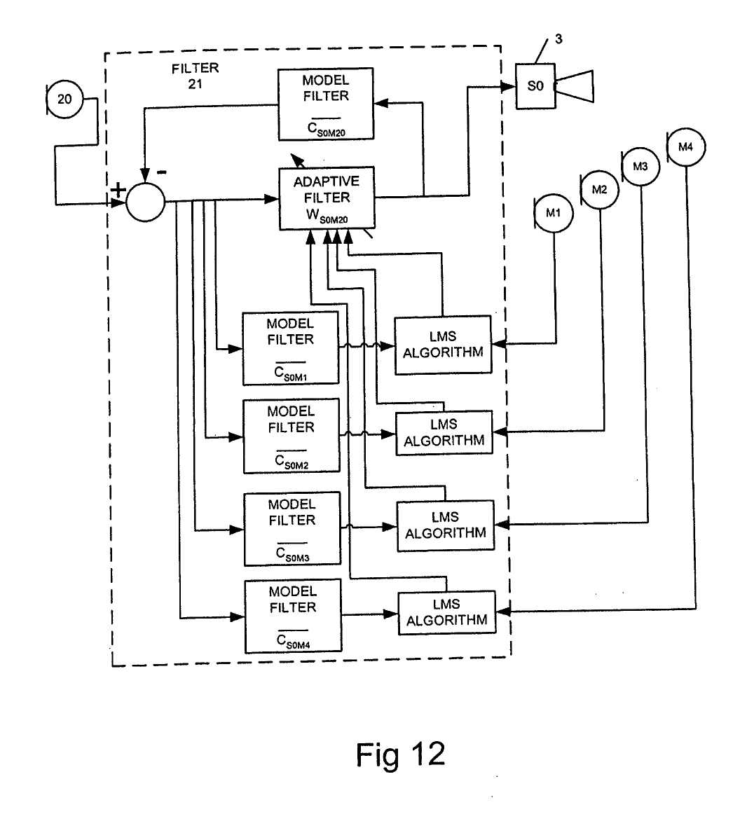

FIG. 12 is a schematic diagram showing in more detail the construction of the filter 21 in the embodiment of FIG. 10, assuming that the acoustic paths between the microphones M1, M2, M3 and M4 and the loudspeaker SO are different;

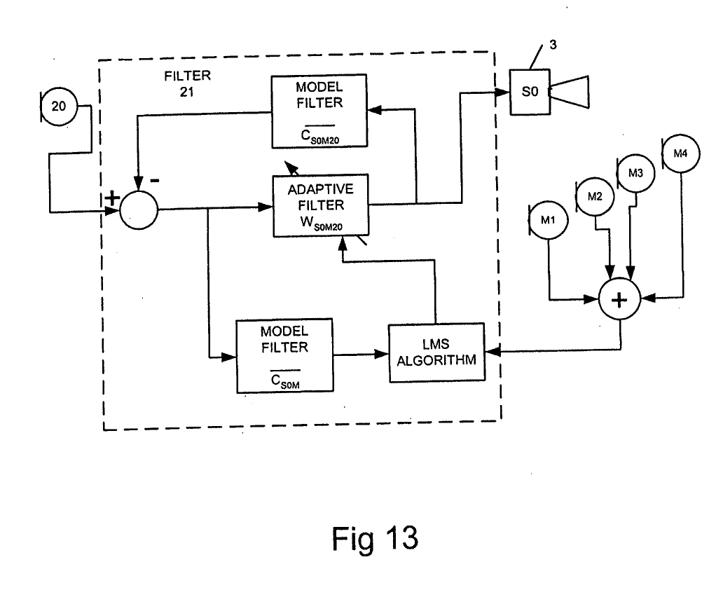

FIG. 13 is a schematic diagram showing in more detail the construction of the filter 21 in the embodiment of FIG. 10, assuming that the acoustic paths between the microphones M1, M2, M3 and M4 and the loudspeaker S0 are the same; and

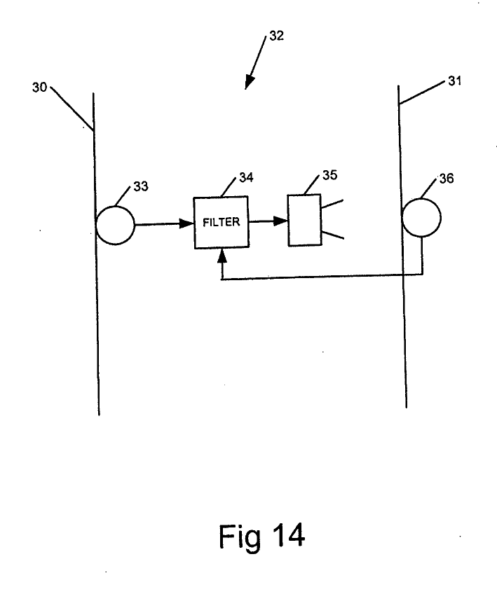

FIG. 14 is a schematic diagram of an active noise control unit for generating a drive signal for an actuator using feedforward and feedback control in accordance with a further embodiment of the present invention.

FIGS. 1 and 2 are diagrams illustrating an embodiment of the present invention in which actuators 3 and sensors 2 are arranged in two interspersed arrays on a panel 1. Each actuator 3 provides a physical actuation which results in an acoustic disturbance in antiphase with undesired acoustic disturbances in order to attenuate the acoustic disturbance. In accordance with this embodiment and the following embodiments of the present invention, the actuators 3 can comprise any form of physical or acoustic actuator such as a loudspeaker, a vibrator, a piezoelectric element for mounting on a panel, or a bending wave generator for generating bending waves in a panel.

The sensors 2 in this embodiment and in the following embodiments can comprise any suitable sensors for sensing acoustic disturbances such as a microphone, an accelerometer, a bending wave detector, or a piezoelectric sensor.

A bending wave detector in an embodiment of the present invention can comprise a device for detecting the bending of the interior or exterior panel. The bending of the interior and exterior panel can cause the generation of noise within the cabin of the vehicle. Noise will be generated when a panel bends at half or a multiple of half the wavelength of the sound. Thus a bending wave detector needs to be tuned to detect these bending waves. The bending wave detector can thus be a strip arranged along the length of the panel at the same length as the length of the panel capable of bending.

The actuator array and the sensor array are provided on a panel 1 in this embodiment so as to provide physical support for the array of actuators and sensors. The panel is not however essential for the embodiment of the present invention.

FIG. 2 shows the provision of the panel 1 as an interior wall of a vehicle cabin. The actuators 2 and the sensors 3 are arranged to lie within a cavity 4 formed between the interior wall 1 and an exterior wall 5. This structure is a typical structure found in vehicles such as aircraft. In such situations the environment outside the exterior wall 5 is extremely noisy and it is desirable to provide a quiet environment in the interior of the cabin adjacent the interior wall 1. Thus, the actuator array and the sensor array provided within the cavity 4 function to attenuate noise propagating across the cavity 4 and along the cavity 4.

In the embodiment of FIGS. 1 and 2, each actuator 3 is controlled using control means (not shown) in dependence upon acoustic disturbances detected by neighbouring sensors 2. In this embodiment there are four neighbouring sensors 2 for each actuator 3. Thus, each “cell” of the noise attenuation system can be considered to comprise a single actuator 3 and four sensors 2. Each sensor 2 is a member of four adjacent cells. Thus each cell is not independent and there is cross talk between cells.

Although this embodiment illustrates the cell arrangement as being square, i.e. each actuator 3 having four sensors and four actuators adjacent to it, the present invention is applicable to any regular or irregular cell arrangement. For example, a hexagonal cell system could be used in which there are six sensors 2 and six actuators 3 adjacent to each actuator 3. Alternatively an irregular grid could be set up in which each actuator 3 has between two and 6 sensors 2 adjacent to them depending on their location.

Although in this embodiment the array of actuators and sensors are shown as a line adjacent to or on the interior wall 1, whilst this provides for the highest degree of noise cancellation, the present invention encompasses the positioning of the array of sensors and actuators anywhere within the cavity 4 or even on the exterior wall 5 since such placement will still provide an improved noise cancelling system compared to the prior art.

FIG. 3 is a schematic diagram of a unit or cell for carrying out noise attenuation in the arrangement of FIGS. 1 and 2. In the embodiment of FIG. 3 the sensors comprise four microphones 2a, 2b, 2c and 2d. The outputs of each microphone are amplified by corresponding amplifiers 6a, 6b, 6c and 6d. The amplified outputs are then summed by a summation unit 7 before being input to a filter 8. The filter 8 performs a function of inversion and appropriate adaption in order to shape the summed signal to an appropriate drive signal for causing noise attenuation. The signal output from the filter 8 is amplified by an amplifier 9. The amplifier 9 preferably comprises a switch mode power amplifier since these produce less heat which is a problem to dissipate in certain environments such as in an aircraft. The output of the amplifier 9 is a drive signal to the actuator 3 which in this embodiment comprises a loudspeaker.

This embodiment thus provides a feedback control system in which the residual noise detected by neighbouring sensors, e.g. microphones, is summed and used to control the generation of cancelling noise by the actuator, i.e. the loudspeaker 3. Since each sensor belongs to a number of cells and thus controls the generation of noise attenuating signals by a plurality of actuators, noise attenuation control is not limited locally.

In the embodiment of FIG. 3, the summation performed by the summation unit 7 can comprise a weighted sum in which any one of the outputs of the sensors can be given a desired weighting.

Thus in this embodiment of the present invention, the sensors 2 will sense acoustic disturbances within the cavity 4 and the sensors adjacent to an actuator 3 will cause the generation of cancelling noise in the cavity 4. Thus, in this way, the array of actuators and sensors can be considered to be a noise attenuating “blanket” e.g. covering the interior wall of the vehicle in order to attenuate noise propagating across and along the cavity 4. The arrangement acts to reduce the noise within the cavity 4, thereby reducing the noise passing across the interior wall 1 into the cabin of the vehicle.

Another embodiment of the present invention will now be described with reference to FIGS. 4 and 5 in which each cell comprising an actuator and a plurality of sensors, i.e. in this embodiment four sensors, operates using adaptive signal processing which is adapted using the least mean square (LMS) algorithm.

FIG. 4 is a schematic diagram of a cell comprising an actuator, i.e. loudspeaker 3 and four neighbouring sensors 2, i.e. microphones M1, M2, M3 and M4. In this embodiment each microphone 2 provides an output signal x1, x2, x3, x4 which is input to a respective filter W1, W2, W3, W4 to generate a respective drive signal y1, y2, y3 or y4 to drive the loudspeaker 3. Thus the output of each microphone M1, M2, M3 and M4 is used to generate a drive signal in a feedforward manner. Thus, in this way, each microphone M1, M2, M3 and M4 acts as a reference source to detect acoustic disturbances propagating towards the loudspeaker 3. Thus each microphone M1, M2, M3 and M4 detects an acoustic disturbance propagating in each respective direction. Each microphone M1, M2, M3 and M4 also acts as a residual sensor for sensing residual vibrations resulting from the combination of the undesired noise and the cancelling noise generated by the loudspeaker 3. Each microphone M1, M2, M3 and M4 generates a respective error signal e1, e2, e3 and e4. Each of these signals is used to adaptively modify the filter coefficients of each filter W1, W2, W3 and W4. This adaption of filter coefficients is performed using the least mean square (LMS) algorithm. The LMS algorithm is well known and is described in the book entitled “Adaptive Signal Processing” by B. Widrow and S. D. Steams. The multi-channel version of the LMS algorithm is also described in WO88/02912 and WO95/26521. In such a multi-channel LMS algorithm the coefficients of the filters comprise a matrix of coefficients.

In the interests of not overcomplicating the diagram FIG. 4 does not illustrate the feedback removal path to remove the feedback effect from the error signals.

FIG. 5 illustrates an example of an adaptive control system for a single channel, i.e. a channel between a microphone and the loudspeaker using the LMS algorithm for adapting the filter coefficients.

As can be seen in FIG. 5, the output of the microphone is split into two outputs. One of the outputs is the error signal e which is input into the LMS algorithm. The other output is used as the reference input x into the adaptive filter W to generate the drive signal y which is amplified by the amplifier 9 and used to drive the loudspeaker 3. Because the reference signal detected by the microphone 2 is corrupted by the output of the loudspeaker 3, a model filter \overline{C}_{F} which models the acoustic path from the loudspeaker to the microphone receives the drive signal y and generates a negative feedback signal f for subtraction from the reference signal input to generate the true reference signal x to be input to the adaptive filter W. The model filter \overline{C}_{F} comprises the feedback removal filter in the feedback removal path. The corrected reference signal x is also input into a copy of the model filter \overline{C} to generate a time aligned reference signal r for correlation with the error signal e within the LMS algorithm. The LMS algorithm modifies the coefficients of the adaptive filter W to adapt the drive signal in order to minimize the sum of the squares of the error signals detected by the microphones 2.

The single channel algorithm illustrated in FIG. 5 comprises the well-known virtual earth circuit as for example disclosed in WO09/12579.

As mentioned earlier, FIG. 5 only illustrates a single channel from a single microphone. There are, however, in the embodiment illustrated in FIGS. 1 and 2, four microphones and thus four channels and the adaptive filter W comprises an array of w₁₁, w₁₂, w₁₃ and w₁₄ coefficients denoting a set of coefficients for the channel from each microphone to the loudspeaker. Similarly, the model filter \overline{C} comprises the coefficients c₁₁, c₁₂, c₁₃ and c₁₄.

Thus, in this embodiment of the present invention the four nearest neighbour sensors are used together with four sets of filter coefficients to generate a drive signal for the actuator.

In the embodiment of FIG. 5, the amplifier 9 preferably comprises a switched mode amplifier which provides low heat dissipation. This is particularly advantageous in the use in an aircraft where heat dissipation is a problem since the addition of heat sinks to amplifiers increases the weight of the amplifier which is clearly undesirable.

Another embodiment of the present invention will now be described with reference to FIGS. 6 to 9. This embodiment of the present invention is a modification of the embodiment of FIGS. 4 and 5. In this embodiment of the present invention, not only are the nearest neighbour microphones used to generate the drive signal for the loudspeaker, also the output of the nearest neighbour loudspeakers are taken into consideration as reference signals indicative of noise propagating across the panel. This is so because the drive signals for a neighbouring loudspeaker will be dependent upon the noise to be cancelled as calculated for the cell in which the loudspeaker belongs, i.e. a neighbouring cell.

FIG. 6 only illustrates the direct drive filter paths and for simplicity the adaption paths have been omitted (i.e. the dashed lines of FIG. 4). Also the feedback removal paths are not shown. It can thus be seen in FIG. 6 that the output of each loudspeaker affects the output of a neighbouring loudspeaker.

FIG. 7 illustrates the interrelationship of the drive signals of loudspeakers in more detail. It will be apparent that the arrangement of FIG. 7 is similar to the arrangement of FIG. 5 except that the output of the adaptive filter for the loudspeaker S0 is also output as reference signals for the four neighbouring loudspeakers S1, S2, S3 and S4. These signals are input to adaptive filters W_{S0S1}, W_{S0S2}, W_{S0S3} and W_{S0S4} in order to generate a signal to modify the drive signal of the adjacent loudspeakers. The drive signal for the loudspeaker S0 is modified by summation with signals output from the adaptive filters W_{S1S0}, W_{S2S0}, W_{S3S0} and W_{S4S0} in a summation unit 10. Drive signals for the neighbouring loudspeakers S1, S2, S3 and S4 are used as the inputs to the filters W_{S1S0}, W_{S2S0}, W_{S3S0} and W_{S4S0}. The summation unit 10 performs summation (or weighted summation) of the drive signals to generate a drive signal for input to the amplifier 9 to drive the loudspeaker S0. Thus in this embodiment of the present invention, the generation of cancelling acoustic noise in a cell is modified in accordance with the generation of cancelling acoustic noise in adjacent cells.

FIG. 8 is a diagram illustrating the details of the feedforward filters W_{SjS0} in the embodiment of FIG. 6. FIG. 8 illustrates one of the filters W_{SjS0} where j=1 to 4. The filter adaptively generates a feedforward signal from the drive signal for loudspeaker S0 from the drive signal from an adjacent loudspeaker Sj. The 4 microphones M1, M2, M3 and M4 adjacent the loudspeaker S0 provide for the error detection which is fed into the LMS algorithm. Time alignment of the error signal with the drive signal from the adjacent loudspeaker Sj is achieved by feeding the drive signal from the adjacent loudspeaker Sj through a corresponding model filter C_{SjMi} where i=1 to 4. This assumes that the acoustic path between a loudspeaker Sj and each of the microphones M1, M2, M3 and M4 is different.

FIG. 9 is a diagram illustrating the adaptive filter W_{SjS0} where it is assumed that the acoustic path between the loudspeaker Sj and the microphones M1, M2, M3 and M4 is the same. In this embodiment a single model filter C_{SjM} is used and the outputs of the microphones are summed before being input into the LMS algorithm.

Thus in this embodiment of the present invention, adaptive filters provide for a feedforward and feedback circuit, i.e. a digital virtual earth circuit, between the adjacent microphones and loudspeakers. Also, adaptive filters provide for a feedforward adaptive signal between adjacent loudspeakers.

It will thus be apparent from the embodiments described with reference to FIGS. 4 to 9 that a noise cancelling surface or plane can be provided which can act to cancel noise impinging on it, i.e. crossing over the cavity, but can also act efficiently to cancel noise propagating across it in view of the feedforward and feedback nature of the configuration of sensors and actuators, i.e. the interspersed arrays.

FIGS. 10 and 11 illustrates a further embodiment of the present invention which is a further modification of the embodiment of FIGS. 6 to 9. This embodiment provides a further feedforward path across the cavity 4. In this embodiment a feedforward sensor 20 associated with the actuator S0 of a cell is arranged across the cavity towards the exterior wall 5. A filter 21 receives the output of the feedforward sensor 20 and this is used as a feedforward signal to drive the actuator. The filter 21, although shown as a separate entity, in fact comprises a set of filter coefficients for the feedforward path. Thus the drive circuit for each actuator will include a further set of filter coefficients and a further input signal from the feedforward sensor 20.

FIG. 12 is a diagram illustrating the details of the filter 21 in more detail. The filter 21 includes an adaptive filter W_{S0M20} for receiving the output of the microphone 20 after it has been corrected for the feedback and for generating the output drive signal to the loudspeaker S0. The feedback correction path is provided in this embodiment by the model filter \overline{C_{S0M20}} for filtering the drive signal to generate a feedback signal for subtraction from the output of the microphone 20. Thus the input to the adaptive filter W_{S0M20} is corrected to remove any influence from the output of the loudspeaker S0.

The microphones M1, M2, M3 and M4 adjacent to the loudspeaker S0 provide for error sensing and the output of the microphones M1, M2, M3 and M4 are input into corresponding algorithms within the filter 21. Correlation signals are input into the LMS algorithms by inputting the feedback corrected signal from the microphone through corresponding model filters \overline{C_{S0M1}}, \overline{C_{S0M2}}, \overline{C_{S0M3}} and \overline{C_{S0M4}}. Thus the LMS algorithm performs adaption of the adaptive filter W_{S0M20}. This embodiment assumes that the acoustic path between each of the microphones M1, M2, M3 and M4 and the loudspeaker S0 are different.

FIG. 13 illustrates an alternative embodiment in which it is assumed that the acoustic paths between the microphones M1, M2, M3 and M4 and loudspeaker S0 are the same. In this embodiment only a single model filter \overline{C_{S0M}} is required and the outputs of the microphones M1, M2, M3 and M4 are summed before being input into the LMS algorithm to be used for adapting the adaptive filter W_{S0M20}.

The embodiments of FIGS. 12 and 13 comprise standard feedforward adaptive filtering using the LMS algorithm.

Although FIG. 10 illustrates only a single feedforward sensor, preferably a number of these are provided, one to an actuator. In one embodiment each actuator can be provided with a corresponding feedforward sensor to provide a feedforward path for acoustic disturbances propagating across the cavity.

Thus in this embodiment of the present invention, the two-dimensional array of actuators and sensors providing for feedforward paths in all directions in addition to feedback paths are supplemented by an additional feedforward path configuration comprised of a number of feedforward sensors arranged across the cavity 4.

In the embodiments of the present invention described so far, the array of actuators and sensors can be arranged within the cavity or on the interior wall (or even on the exterior wall when no feedforward sensor or sensors 20 are used). They can be mounted on a panel 1 which need not be the interior panel of the vehicle. The panel 1 can comprise a suitable mounting medium for the array of sensors and actuators. The panel 1 can then be placed within the cavity. The actuators and sensors are preferably arranged within the cavity near to the interior wall so as to attenuate the noise within the cavity near the interior wall and to enable the use of feedforward sensors 20.

FIG. 14 is a schematic diagram of a further embodiment of the present invention which uses a feedforward and feedback signal for controlling an actuator 35 to attenuate noise propagating across the cavity 32 between an exterior wall 30 and an interior wall 31 of a vehicle. A reference sensor 33 is arranged towards the exterior wall 30 in order to detect acoustic disturbances propagating towards the interior wall 31. The reference signal from the reference sensor 33 is input to a filter 34 in order to generate a drive signal for an actuator to generate noise cancelling vibrations to cancel out noise in the vicinity of or towards the interior wall 31. Towards the interior wall 31 there is arranged a residual sensor 36 for detecting residual noise or vibrations. The residual signal produced by the residual sensor 36 is used to adapt the filter 34 to modify the control signal to the actuator 35 to reduce the residual noise detected by the residual sensor 36.

Thus in this embodiment of the present invention, the reference sensor 33 provides a feedforward signal detecting vibrations or noise which will cause acoustic disturbances to propagate across the cavity 32. The residual sensor 36 will provide a feedback signal detecting how successful the system is in cancelling or reducing the noise in the vicinity of or towards the interior wall 31.

In this embodiment of the present invention, the reference sensor 33 can comprise any suitable sensor such as a microphone, a piezoelectric element mounted on the exterior wall 30, a bending wave detector mounted on the exterior wall 30, or an accelerometer for example. Similarly, the residual sensor can comprise a microphone, a piezoelectric device mounted on the interior wall 31, a bending wave sensor mounted on the interior wall 31, or an accelerometer mounted on the interior wall 31. The actuator 35, although illustrated as comprising a loudspeaker, can comprise any suitable actuator such as a vibrator mounted on the interior wall 31, a piezoelectric element mounted on the interior wall 31, or a bending wave generator mounted on the interior wall 31.

All of the embodiments described hereinabove are particularly suited for use in an aircraft where there is a significant amount of undesirable noise exterior to the aircraft cabin which it is desired to prevent reaching the aircraft cabin. The embodiments of the present invention provide a system which can conveniently be mounted within the cavity between the exterior fuselage and the cabin wall, thereby avoiding encroachment on the cabin space. The system of providing a noise cancelling arrangement within the cavity also acts to try to prevent or reduce the noise entering the cabin.

In the arrangement of the embodiments of the present invention described hereinabove, the filtering to control and generate the drive signal for each actuator can either be done locally, i.e. in independent controllers, or in a central controller separately for each actuator. The filtering or controller for each actuator can comprise suitable hardware dedicated for the purpose, or programmed hardware. Preferably, microprocessor means are used for providing the digital filtering. Separate microprocessors can be provided for each actuator or in localized processing units for a number of actuators. Alternatively, a large central microprocessing arrangement can be provided for performing the filtering operation. Such a system is disclosed for example in WO88/02912.

Although the present invention has been described hereinabove with reference to specific embodiments, it will be apparent to a skilled person in the art that modifications lie within the spirit and scope of the present invention.

Although the embodiments of the present invention show the array of sensors and actuators lying within a plane which is flat, the array of sensors and actuators can be aligned along any surface which can be curved or shaped according to the interior wall of the cabin so as to provide a noise attenuating “blanket” to attenuate noise reaching the vehicle cabin.

The present invention is intended to encompass the attenuation of any form of noise and vibration including sound and vibration propagating in a solid medium, e.g. panel vibrations.

1. A system for attenuating noise in a cabin of a vehicle, the vehicle having an interior wall defining the cabin and an exterior wall defining a cavity between the interior wall and the exterior wall, the system comprising:
a plurality of actuators arranged in an array within said cavity, on said interior wall, or on said exterior wall for providing a physical actuation resulting in an acoustic disturbance;
a plurality of sensors arranged in an array interspersed said actuators within said cavity, on said interior wall, or on said exterior wall, such that each actuator has a plurality of said sensors adjacent thereto; and
a control system connected to said actuators and said sensors for receiving signals from said sensors indicative of acoustic disturbances and for generating and outputting drive signals to said actuators to reduce the acoustic disturbances, wherein said control system is adapted to generate a drive signal for each actuator using said signals from said sensors adjacent to said actuator.

2. A system according to claim 1, wherein said control system is adapted to perform a sum or weighted sum of said signals from said sensors adjacent to a said actuator to generate said drive signal for said actuator.

3. A system according to claim 2, wherein said control system is adapted to filter each summation.

4. A system according to claim 1, wherein said control system is adapted to perform adaptive filtering of said signals from said sensors adjacent to a said actuator to generate said drive signal for said actuator.

5. A system according to claim 4, wherein said control system is adapted to perform the least mean square algorithm for adapting the filtering.

6. A system according to claim 4, wherein said control system is adapted to generate said drive signal for a said actuator using the drive signals for adjacent said actuators.

7. A system according to claim 6, wherein said control system is adapted to perform adaptive filtering on said drive signals for adjacent said actuators to generate said drive signal for said actuator.

8. A system according to claim 4, wherein said array of actuators and said array of sensors are arranged in said cavity or on said interior wall, the system including at least one reference sensor arranged away from said array of actuators and said array of sensors towards said exterior wall for providing at least one reference detection indicative of acoustic disturbances propagating across said cavity towards said array of actuators and said array of sensors, wherein said control system is adapted to use said at least one reference detection in the generation of said drive signal for at least one said actuator.

9. A system according to claim 8, including an array of said reference sensors arranged towards said exterior wall substantially parallel to said array of actuators and said array of sensors.

10. A system according to claim 1, wherein said control system is adapted to use said signals from said adjacent sensors as feedback and feedforward control signals in the generation of said drive signal for a said actuator.

11. A system for attenuating noise in a cabin of a vehicle, the vehicle having an interior wall defining the cabin and an exterior wall defining a cavity between the interior wall and the exterior wall, the system comprising:
a plurality of actuators arranged in an array on a panel for placement in said cavity for providing a physical actuation resulting in an acoustic disturbance;
a plurality of sensors arranged in an array interspersed said actuators on said panel, such that each actuator has a plurality of said sensors adjacent thereto; and
a control system for connection to said actuators and said sensors for receiving signals from said sensors indicative of acoustic disturbances and for generating and outputting drive signals to said actuators to reduce the acoustic disturbances, wherein said control system is adapted to generate each drive signal for each actuator using said signals from said sensors adjacent to said actuator.

12. A system according to claim 11, wherein said control system is adapted to perform a sum or weighted sum of said signals from said sensors adjacent to a said actuator to generate said drive signal for said actuator.

13. A system according to claim 12, wherein said control system is adapted to filter each summation.

14. A system according to claim 11, wherein said control system is adapted to perform adaptive filtering of said signals from said sensors adjacent to a said actuator to generate said drive signal for said actuator.

15. A system according to claim 14, wherein said control system is adapted to perform the least mean square algorithm for adapting the filtering.

16. A system according to claim 14, wherein said control system is adapted to generate said drive signal for a said actuator using the drive signals for adjacent said actuators.

17. A system according to claim 16, wherein said control system is adapted to perform adaptive filtering on said drive signals for adjacent said actuators to generate said drive signal for said actuator.

18. A system according to claim 14, including at least one reference sensor for arrangement away from said array of actuator and said array of sensors towards said exterior wall for providing at least one reference detection indicative of acoustic disturbances propagating across said cavity towards said array of actuators and said array of sensors, wherein said control system is adapted to use said at least one reference detection in the generation of said drive signal for at least one said actuator.

19. A system according to claim 18, including an array of said reference sensors for arrangement towards said exterior wall substantially parallel to said array of actuators and said array of sensors.

20. A system according to claim 11, wherein said control system is adapted to use said signals from said adjacent sensors as feedback and feedforward control signals in the generation of said drive signal for a said actuator.

21. A system for attenuating noise in a cabin of a vehicle, the vehicle having an interior wall defining the cabin and an exterior wall defining a cavity between the interior wall and the exterior wall, the system comprising:
a plurality of actuators arranged in said cavity for providing a physical actuation resulting in an acoustic disturbance;
a plurality of sensors arranged in said cavity for providing signals indicative of acoustic disturbances; and
control means for generating a drive signal for each actuator using said signals from a plurality of said sensors adjacent to each respective said actuator.

22. A system according to claim 21, wherein said sensors comprise bending wave detectors.

23. A system according to claim 22, wherein said bending wave detectors are configured to detect bending waves that will cause the generation of noise in the cabin of the vehicle.

24. A system according to claim 21, wherein said sensors comprise accelerometers.

25. A system according to claim 21, including at least one reference sensor arranged to provide at least one reference signal indicative of acoustic disturbances entering said cavity, wherein said control means is adapted to use a said reference signal in the generation of each drive signal for each actuator.

26. A system according to claim 21, wherein said control means is adapted to generate said drive signals for adjacent said actuators using said signals from some of the same said sensors.

27. A system according to claim 21, wherein said control means is adapted to perform a sum or weighted sum of said signals from said sensors in the generation of said drive signals.

28. A system according to claim 21, wherein said control means comprises a plurality of independent controllers, one for each actuator.

29. A system for attenuating noise in a cabin of a vehicle, the vehicle having an interior wall defining the cabin and an exterior wall defining a cavity between the interior wall and the exterior wall, the system comprising:
at least one reference detector arranged on or towards said exterior wall for providing at least one reference signal indicative of acoustic disturbances;
at least one actuator arranged on or towards said interior wall for providing at least one physical actuation resulting in at least one acoustic disturbance;
at least one residual detector arranged on or towards said interior wall for providing at least one residual signal indicative of residual acoustic disturbances; and
a control system for using said at least one reference signal and said at least one residual signal in the generation of at least one drive signal for said at least one actuator.

30. A system for attenuating noise in a cabin of a vehicle, the vehicle having an interior wall defining the cabin and an exterior wall defining a cavity between the interior wall and the exterior wall, the system comprising:
at least one reference detector for arrangement on or towards said exterior wall for providing at least one reference signal indicative of acoustic disturbances;
at least one actuator for arrangement on or towards said interior wall for providing at least one physical actuation resulting in at least one acoustic disturbance;
at least one residual detector for arrangement on or towards said interior wall for providing at least one residual signal indicative of residual acoustic disturbances; and
a control system for using said at least one reference signal and said at least one residual signal in the generation of at least one drive signal for said at least one actuator.

31. A system according to claim 29, wherein at least one said reference detector and/or at least one said residual detector comprise a bending wave detector.

32. A system according to claim 31, wherein the or each bending wave detector is configured to detect bending waves that will cause the generation of noise in the cabin of the vehicle.

33. A system according to claim 29, wherein at least one said reference detector and/or at least one said residual detector comprise an accelerometer.

34. A system for attenuating noise in a cabin of an aircraft, the aircraft having an interior wall defining the cabin and an exterior fuselage defining a cavity between the interior wall and the exterior fuselage, the system comprising:
at least one actuator arranged in said cavity, on said interior wall, or on said exterior wall for providing at least one physical actuation resulting in at least one acoustic disturbance;
at least one sensor arranged in said cavity, on said interior wall, or on said exterior wall for providing at least one indication of acoustic disturbances;
a control system for using said indications from said at least one sensor to generate at least one control signal for said at least one actuator; and
switch mode power amplifier means for amplifying the or each generated control signal to generate a drive signal for said at least one actuator.

35. A system for attenuating noise in a cabin of a vehicle, the vehicle having an interior wall defining the cabin and an exterior wall defining a cavity between the interior wall and the exterior wall, the system comprising:
at least one actuator for arrangement in said cavity, on said interior wall, or on said exterior wall for providing at least one physical actuation resulting in at least one acoustic disturbance;
a plurality of sensors for arrangement in said cavity, on said interior wall, or on said exterior wall for providing indications of acoustic disturbances; and
a control system for using said indications from said sensors to generate at a signal for the or each actuator, wherein said control system is adapted to use said indications from at least one said sensor as both feedforward signals indicative of at least one propagating acoustic disturbance and feedback signals indicative of at least one residual acoustic disturbance.
